(19) United States
(12) Patent Application Publication
(10) Pub. No.: US 2023/0293514 A1
(43) Pub. Date: Sep. 21, 2023

(54) INJECTABLE DEPOT FORMULATION COMPRISING CARIPRAZINE FREE BASE PARTICLES

(71) Applicant: Anxo Pharmaceutical Co., Ltd., Taipei City (TW)

(72) Inventors: I-Hsiang LIU, Taipei City (TW); Kuei-Hua CHANG, Taipei City (TW); Hua-Jing JHAN, Taipei City (TW); Tse-Hsien CHEN, Taipei City (TW); Chia-Yu SU, Taipei City (TW); Chi-Heng JIAN, Taipei City (TW); Chun-Wei HSU, Taipei City (TW)

(21) Appl. No.: 18/184,947

(22) Filed: Mar. 16, 2023

Related U.S. Application Data
(60) Provisional application No. 63/320,696, filed on Mar. 17, 2022.

(51) Int. Cl. A61K 31/495; A61K 9/00; A61K 47/38; A61K 47/10; A61K 47/32; A61K 47/34; A61K 9/16

(52) U.S. Cl. CPC A61K 31/495; A61K 9/0019; A61K 47/38; A61K 47/10; A61K 47/32; A61K 47/34; A61K 9/16

(57) ABSTRACT

Disclosed herein is an injectable depot formulation and use thereof for treating a mental disorder. The injectable depot formulation comprises cariprazine free base particles and a pharmaceutically acceptable carrier. The cariprazine free base particles have a median particle size by volume (Dv50) ranging from 0.5 μm to 100 μm.

### CROSS-REFERENCE TO RELATED APPLICATION

This application claims the benefit of U.S. Provisional Patent Application No. 63/320696, filed on Mar. 17, 2022, which is incorporated by reference herein in its entirety.

### FIELD

The present disclosure relates to an injectable depot formulation comprising cariprazine, and more particularly to an injectable depot formulation comprising cariprazine free base particles.

### BACKGROUND

Cariprazine is an atypical anti-psychotic, which is classified as a potent dopamine D3 and D2 partial agonist with preferential binding to D3 receptor for the treatment of adult schizophrenia and bipolar disorders. Cariprazine possesses the therapeutic potential superior to other marketed anti-psychotic drugs, and does not trigger severe side effects associated with extrapyramidal symptoms due to its high D3/D2 selectivity. Oral administration of cariprazine effectively prevents patients from orthostatic hypotension, a side effect commonly seen in patients administered with types of other anti-psychotics. Compared to risperidone, patients orally administered with cariprazine received lower PANSS-factor score, reflecting improvement of the predominant negative symptoms of schizophrenia in a 26-week phase 3B clinical trial.

Vraylar® (cariprazine in capsule form) is a prescription medicine which received approval from US FDA in 2015 to be marketed for treatment of schizophrenia, manic or mixed episodes associated with bipolar 1 disorder, depressive episodes associated with bipolar 1 disorder (bipolar depression), and major depressive disorder (MDD), and which is formulated for daily use and immediate release of the drug with the strength of 1.5 mg, 3.0 mg, 4.5 mg, or 6.0 mg, depending on the efficacy and tolerability of individual patient.

The active ingredient of Vraylar® capsule is cariprazine hydrochloride, which has a chemical structure represented by

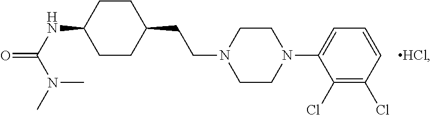 [SMILES: CN(C)C(=O)N[C@H]1CC[C@@H](CCN2CCN(c3cccc(Cl)c3Cl)CC2)CC1]

and which has a chemical name of trans-N-{4-[2-[4-(2,3-dichlorophenyl)piperazine-1-yl]ethyl] cyclohexyl}-N′, N′-dimethylurea hydrochloride. Cariprazine hydrochloride is a non-hygroscopic white to off-white crystalline solid, and extensively produces two active metabolites in liver, i.e., desmethyl-cariprazine (DCAR) and didesmethyl-cariprazine (DDCAR), equipotent to the parent compound cariprazine due to their similarity in in vitro receptor profile, plasma protein binding affinity, as well as the brain penetration.

Injectable depot formulations have been widely applied to the field of antipsychotics, for examples, Flupentixol decanoate (Fluanxol® Depot), Fluphenazine decanoate (Modecate®), Haloperidol decanoate (Haldol®), Zuclopenthixol decanoate (Clopixol® Depot), Aripiprazole monohydrate (Abilify Maintena®), Aripiprazole Lauroxil (Aristada®), Olanzapine pamoate (Zyprexa Relprevv®), Paliperidone palmitate (Invega Sustenna®, Invega Trinza®, Invega Hafyera®), and Risperidone (Risperdal Consta®, Perseris® Kit). These injectable depot formulations exhibit suspended release over a prolonged period of two weeks and up to six months. The long acting efficacy increases the sustained release time and reduces the dosing frequency, thereby improving the patient compliance and lowering the relapse rate of psychotic patients in need of long term treatment.

Long-acting injectable formulations of cariprazine have been developed in the form of non-aqueous suspensions, aqueous suspensions, or microspheres.

U.S. Patent Application Publication No. 20170196855A1 discloses a technology platform applicable to preparing the extended-release formulation of cariprazine dispersed in a non-aqueous liquid vehicle comprising a hydrophobic lipid, a hydrophilic organic solvent, or a combination of both, and an amphiphilic agent. In this invention, a hydrophobic lipid vehicle comprises glyceryl ester of a C6-C24 fatty acid, sesame oil, a hydrophilic organic solvent. The hydrophilic organic solvent comprises polyethylene glycol, propylene glycol, and/or glycerin. The amphiphilic agent comprises sorbitan esters, or polyethoxylated sorbitan esters. In this invention, the high amount of hydrophilic organic solvent used in non-aqueous vehicle might cause irritation-related responses at the site of injection.

Chinese Invention Patent No. 108261394B discloses an injectable aqueous suspension comprising cariprazine hydrochloride as the active pharmaceutical ingredient, which is capable of long-acting and high dosing for at least one week. The long-acting behavior is capable of reducing dosing frequency. However, the cariprazine hydrochloride might cause the formulation to be formed with extremely acidic pH value and high osmolality if excess amounts of sodium hydroxide is used to neutralize the formulation.

Chinese Invention Patent Application Publication No. 112972388A discloses microsphere, microparticle, and nanoparticle formulations of cariprazine dispersed in a polymer matrix material comprising poly(d,l-lactide-glycotide) with a stable sustained release profile without burst release behavior, which commonly occurred at the initial stage of matrix disintegration. In the manufacturing of microsphere formulation of cariprazine, the high cost and scale-up production are the main issues.

The pharmaceutically acceptable salts of cariprazine developed using recrystallization method had been previously reported in U.S. Pat. No. 7,943,621B2. In this patent document, the salt forms of cariprazine were obtained by reacting cariprazine free base with different acids including hydrogen chloride, hydrogen bromide, maleic acid, and methanesulphonic acid in aqueous or non-aqueous solvent system. Among the different salts of cariprazine, cariprazine monohydrochloride salt can be readily isolated in the highest yield and highest purity, which is suitable for industrial scale production. The hydrochloride salts of cariprazine can remain stable in the solid state or aqueous solution at ambient temperature for at least two years. The suitable solubility of the hydrochloride salt enables cariprazine to be immediately released from the oral dosage form, and thus, cariprazine hydrochloride is chosen as the active ingredient in Vraylar® (cariprazine capsules).

However, in the injectable depot formulation, the active ingredient needs to be slowly released from injectable depot formulation at injection site so as to exert long-acting efficacy. As a result, water-soluble hydrochloride salt of cariprazine in solid form might not be suitable to be developed as the injectable depot formulation.

Therefore, those skilled in the art strive to develop an improved injectable depot formulation comprising cariprazine with sustained drug release, reduced dosing frequency, ease of administration, high patient compliance, and reduced risk of adverse effects.

### SUMMARY

Therefore, an object of the present disclosure is to provide an injectable depot formulation which can alleviate at least one of the drawbacks of the prior art.

According to the present disclosure, the injectable depot formulation comprises cariprazine free base particles and a pharmaceutically acceptable carrier. The cariprazine free base particles have a median particle size by volume (Dv50) ranging from 0.5 μm to 100 μm.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 is a graph showing the cumulative amount of cariprazine released at different time points of the dissolution test for injectable depot formulations of Example 1, infra.

FIG. 2 is a graph showing the cumulative amount of cariprazine released at different time points of the dissolution test for injectable depot formulations of Example 2, infra.

FIG. 3 is a graph showing the cumulative amount of cariprazine released at different time points of the dissolution test for injectable depot formulations of Example 3, infra.

FIG. 4 is a graph showing the cumulative amount of cariprazine released at different time points of the dissolution test for injectable depot formulations of Example 4, infra.

FIG. 5 is a graph showing the cumulative amount of cariprazine released at different time points of the dissolution test for injectable depot formulations of Example 5, infra.

FIG. 6 is a graph showing the cumulative amount of cariprazine released at different time points of the dissolution test for the injectable depot formulations of Example 6, infra.

FIG. 7 is a graph showing the mean plasma concentration of cariprazine in the rats versus time following intramuscular administration of the injectable depot formulations of the present disclosure of a first pharmacokinetic study of Example 7, infra.

FIG. 8 is a graph showing the mean plasma concentration of cariprazine in the rats versus time following oral administration of Vraylar® capsule of a second pharmacokinetic study of Example 7, infra.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The applicants initially performed a solubility study on cariprazine hydrochloride salt and cariprazine free base using shake-flask method, and discovered that the water solubility of cariprazine hydrochloride salt (>5 mg/mL) was much higher than that of the cariprazine free base (0.006 mg/mL). In addition, cariprazine hydrochloride salt is a water soluble salt, meaning that certain amounts of cariprazine were already solubilized in the injectable depot formulation, possibility resulting in potential dose dumping issue in patients. Considering that cariprazine free base with lower aqueous solubility can exhibit better sustained-release behavior, cariprazine free base is chosen as the active ingredient in injectable depot formulation.

It is believed that poorly soluble salts of cariprazine possess relatively low aqueous solubility compared to the water soluble salts of cariprazine. Preparation of poorly soluble salts of cariprazine have several merits such as ease of synthesis in a single crystalline form, less risk of solvate formation stemming from organic solvents used during synthesis, readily isolable in high yield and purity, and even lower aqueous solubility than the free base form of cariprazine under certain circumstances. However, some adverse clinical events have been monitored in patients receiving the salt-based long acting formulation of cariprazine.

An example of a poorly soluble salt developed as an injectable depot formulation is olanzapine pamoate, which is the active ingredient of Zyprexa Relprevv® used in the treatment of schizophrenia. Some patients intramuscularly administered with Zyprexa Relprevv® once every 2 to 4 weeks have post-injection delerium/sedation syndrome (PDSS) due to a sudden increase in plasma level of olanzapine. In vitro solubility study revealed that patients administered with olanzapine pamoate had solubilization enhancement in plasma which is 18-fold higher compared to that in pH 7.6 buffer, which is used to simulate the environment of muscular tissue. It is possible that accidental infiltration of blood to the injectable depot at the injection site resulted in unexpectedly rapid solubilization of olanzapine pamoate, causing irreversible dissociation of olanzapine pamoate to form a parent drug olanzapine and a pamoic acid, which results in overdose of olanzapine. Since an aberrant increase in plasma drug concentration upon injection of salt-forming formulation is to be avoided, the injectable depot formulation of the present disclosure comprises the free base form of cariprazine as the active ingredient.

Therefore, the present disclosure provides an injectable depot formulation comprising cariprazine free base particles and a pharmaceutically acceptable carrier. The cariprazine free base particles have a median particle size by volume (Dv50) ranging from 0.5 μm to 100 μm.

In certain embodiments, the cariprazine free base particles have a median particle size by volume (Dv50) ranging from 2 μm to 40 μm, 5 μm to 40 μm, 10 μm to 40 μm, or 20 μm to 40 μm.

In certain embodiments, the cariprazine free base particles have a median particle size by volume (Dv50) ranging from 2 μm to 25 μm, 5 μm to 25 μm, or 10 μm to 25 μm.

As used herein, the term “injectable depot formulation” is used to encompass a formulation comprising an active ingredient in a solid form (e.g., powders, cakes, granules, etc.) that does not comprise water for injection, and a formulation comprising the active ingredient in the form of an aqueous suspension that comprises water for injection.

As used herein, the term “median particle size by volume” of X μm means that 50% of the volume of the cariprazine free base particles consists of particles having an equivalent diameter of less than X μm.

In certain embodiments, the cariprazine free base particles are present in an amount ranging from 4 wt % to 65 wt %, based on a total weight of the injectable depot formulation.

In certain embodiments, the pharmaceutically acceptable carrier may comprise an agent that is at least one of a suspending agent, a buffering agent, and a tonicity agent.

In certain embodiments, the suspending agent is present in an amount ranging from 0.2 wt % to 10 wt %, based on the total weight of the injectable depot formulation.

As used herein, the term “suspending agent” refers to an excipient added to disperse systems in order to maintain particulate ingredients in suspension or to avoid other forms of physical instability. The most common suspending agents are aqueous polymers which comprises methylcellulose, sodium carboxymethyl cellulose, and hydroxypropylmethylcellulose.

In certain embodiments, the suspending agent comprises a cellulose derivative, povidone, and polyethylene glycol. In an exemplary embodiment, the suspending agent is the cellulose derivative.

In certain embodiments, the cellulose derivative comprises carboxymethyl cellulose, sodium carboxymethyl cellulose, and methyl cellulose. In an exemplary embodiment, the cellulose derivative is sodium carboxymethyl cellulose.

In certain embodiments, the buffering agent is present in an amount ranging from 0.01 wt % to 2 wt %, based on the total weight of the injectable depot formulation.

As used herein, the term “buffering agent” refers to salts of a weak acid or a weak base that are used in pharmaceutical solutions to control the pH of a formulated product.

In certain embodiments, the buffering agent comprises sodium phosphate monobasic, disodium hydrogen phosphate, citric acid, sodium citrate, or combinations thereof. In an exemplary embodiment, the sodium phosphate monobasic is sodium hydrogen phosphate monohydrate, and the disodium hydrogen phosphate is disodium hydrogen phosphate anhydrous.

In certain embodiments, the tonicity agent is present in an amount ranging from 0.01 wt % to 6.5 wt %, based on the total weight of the injectable depot formulation.

As used herein, the term “tonicity agent” refers to an agent used to control the osmotic pressure of a solution. The tonicity agent may be classified as dextrose, glycerin, mannitol, potassium chloride, or sodium chloride.

In certain embodiments, the tonicity agent comprises mannitol, glucose, sucrose, trehalose, and sodium chloride.

In certain embodiments, the pharmaceutically acceptable carrier further comprises a wetting agent.

As used herein, the term “wetting agent” refers to an agent which can reduce the surface tension of a liquid, allowing the wetting agent to spread across and penetrate the surface of a solid. The term “wetting agent” can be used interchangeably with the term “surfactant” that are akin to amphiphilic.

In certain embodiments, the wetting agent is present in an amount ranging from 0.01 wt % to 3 wt %, based on the total weight of the injectable depot formulation.

In certain embodiments, the wetting agent comprises polysorbate, poloxamer, lecithin, sorbitan monolaurate, and polyoxyethylene fatty acid esters. In an exemplary embodiment, the wetting agent is poloxamer. In another exemplary embodiment, the wetting agent is lecithin.

In certain embodiments, the pharmaceutically acceptable carrier further comprises a pH-adjusting agent.

In certain embodiments, the pH-adjusting agent is present in an amount ranging from 0.01 wt % to 1 wt %, based on the total weight of the injectable depot formulation.

In certain embodiments, the pH-adjusting agent comprises sodium hydroxide and hydrogen chloride.

The present disclosure also provides another injectable depot formulation comprising cariprazine free base particles and a pharmaceutically acceptable carrier. The cariprazine base particles have a Dv50 ranging from 0.5 μm to 100 μm. The pharmaceutically acceptable carrier comprises at least one of a suspending agent, a buffering agent, a tonicity agent, and combinations thereof. The suspending agent may be selected from cellulose derivative, povidone, and polyethylene glycol; the buffering agent may be selected from sodium phosphate monobasic, disodium hydrogen phosphate, citric acid, sodium citrate, and combinations thereof; and the tonicity agent may be selected from mannitol, glucose, sucrose, trehalose, and sodium chloride.

In an exemplary embodiment, the suspending agent is sodium carboxymethyl cellulose; the buffering agents are disodium hydrogen phosphate monohydrage and disodium hydrogen phosphate anhydrous; and the tonicity agent is mannitol.

The present disclosure also provides a method for treating psychosis, cognitive impairment accompanying schizophrenia, bipolar disorder, acute mania, mild-to-moderate cognitive deficits, dementia, psychotic states associated with dementia, psychotic depression, mania, paranoid and delusional disorders, dyskinetic disorders, neuroleptics-induced parkinsonism, tardive dyskinesia, eating disorders, attention deficit disorder, hyperactivity disorders in children, depression, anxiety, sexual dysfunction, sleep disorders, emesis, aggression, autism, major depressive disorder, or drug abuse, which comprises the step of administering the aforesaid injectable depot formulation to a subject in need thereof.

The present disclosure will be described by way of the following examples. However, it should be understood that the following examples are intended solely for the purpose of illustration and should not be construed as limiting the present disclosure in practice.

### EXAMPLES

### Example 1. Evaluation of the Effect of Different Median Particle Size by Volume (Dv50) of Cariprazine Free Base Particles on in Vitro Dissolution of Injectable Depot Formulation

An injectable depot formulation comprising cariprazine free base particles (serving as the active ingredient) was prepared using the recipe shown in

Table 1 below.

TABLE 1

| Components of IDF of cariprazine | Amount | |
|---|---|---|
| Cariprazine free base particles | 7.56 | g |
| Sodium carboxymethyl cellulose | 1.4976 | g |
| Mannitol | 7.4880 | g |
| Sodium dihydrogen phosphate monohydrate | 0.1043 | g |
| Disodium hydrogen phosphate anhydrous | 0.1482 | g |
| Water (for injection) | 120 | mL |

To be specific, a vehicle solution was prepared by mixing excipients, i.e., sodium carboxymethylcellulose, mannitol, sodium dihydrogen phosphate monohydrate, disodium hydrogen phosphate anhydrous using a mechanical stirrer at a speed ranging from 300 to 500 rpm for 3 hours, and then cariprazine free base particles (in solid powder form) were added into the vehicle solution, followed by mixing at 200 rpm for 18 hours at 25° C. so as to obtain a milky suspension. Thereafter, the milky suspension was subjected to homogenization at 11000 rpm for 10 minutes to obtain a primary suspension. Afterwards, the primary suspension was subjected to a wet ball milling process using an agitator bead mill at 15° C. so as to reduce the size of the cariprazine free base particles, thereby obtaining suspensions comprising cariprazine free base particles with target particle sizes, namely, suspensions of experimental groups D1 to D5. Then, laser scattering analysis using a particle size analyzer (Malvern Mastersizer 3000) was applied to determine the particle size distributions of the cariprazine free base particles, which were shown as volumetric diameters of Dv10, Dv50 and Dv90. The results are shown in Table 2 below.

The concentration of cariprazine free base particles in each group was determined by HPLC. The thus determined concentration of cariprazine free base particles (see Table 2) was divided by theoretical drug concentration thereof, so as to obtain assay (in %) (see Table 2). The suspensions of each group was filled into a glass vial, stoppered, sealed, and then subjected to moist heat sterilization at 121° C. for 40 minutes (overkill cycle), thereby obtaining the injectable depot formulations of Experimental groups D1 to D5 each in the form of an aqueous ready-to-use suspension. In addition, a well-known reference listed drug (RLD), i.e., Vraylar® oral capsule comprising the cariprazine hydrochloride salt, was used as a control, and named as Comparative group 1.

TABLE 2

| | Concentration | | | | |
|---|---|---|---|---|---|
| | of | Particle size distribution | | | |
| | cariprazine | Dv10 | Dv50 | Dv90 | Assay |
| Suspension | (mg/mL) | (μm) | (μm) | (μm) | (%) |
| Experimental group D1 | 52.5 | 0.76 | 2.17 | 4.42 | 104.0 |
| Experimental group D2 | 50.0 | 2.70 | 8.40 | 28.30 | 101.0 |
| Experimental group D3 | 52.5 | 4.17 | 9.59 | 33.00 | 102.5 |
| Experimental group D4 | 50.0 | 8.36 | 22.20 | 75.50 | 95.1 |
| Experimental group D5 | 50.0 | 7.40 | 40.30 | 125.00 | 99.2 |

Referring to Table 2, the Dv50 of the cariprazine free base particles in the injectable depot formulations of Experimental groups D1 to D5 are approximately 2 μm, 8 μm, 10 μm, 20 μm and 40 μm, respectively, and the assay values thereof ranges from 95% to 105%.

The injectable depot formulations of Experimental groups D1 to D5 and the Vraylar® oral capsule of Comparative group 1 was subjected to a dissolution test using the conditions shown in Table 3 below. The concentration of cariprazine free base particles in the Experimental groups D1 to D5 and the Vraylar® oral capsule of Comparative group 1 were analyzed by HPLC method as shown in Table 4, so as to determine the cumulative amount of cariprazine released (in %) in each group.

TABLE 3

| Parameter | Setting |
|---|---|
| Apparatus | USP Apparatus 2 (Paddle) |
| Rotation speed | 50 rpm |
| Dissolution medium | 0.25% SDS |
| Temperature | 37.0 ± 0.5° C. |
| Sample introduction | IDF of cariprazine |
| Sample drawn | 5 mL |
| Sampling mode | Automatic |
| Sampling time | 3, 6, 10, 15, 30, 60, 120 min |
| Sample treatment | Immediately filter through a filter having |
| prior to HPLC | pore size of 0.22 μm |
| analysis | |

TABLE 4

| Parameter | Setting |
|---|---|
| Column | YMC triat C18, 250 × 4.6 mm, 5 μm |
| Detector | UV at 215 nm |
| Flow Rate | 1.2 mL/min |
| Mobile Phase (MP) | MPA: ACN |
| | MPB: 1.74 g K₂HPO₄/5% diethylamine in 1 L |
| | water, pH 6.5 |
| Elution ratio | MPA:MPB = 60:40 |
| Injection Volume | 20 μL |
| Column temperature | 25° C. |
| Sampler temperature | 25° C. |
| Run Time | 12 min |

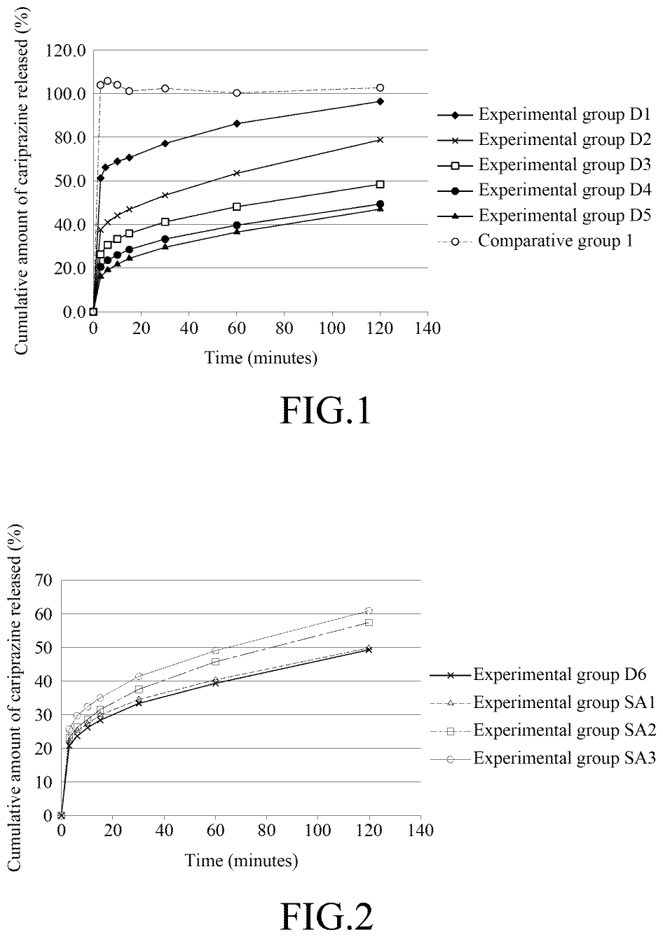

The results are shown in FIG. 1, and Tables 5 and 6.

TABLE 5

| | | | Experimental group D1 | | Experimental group D2 | |
|---|---|---|---|---|---|---|
| | Comparative group 1 | | (Dv50 = 2 μm) | | (Dv50 = 8 μm) | |
| | Cumulative | | Cumulative | | Cumulative | |
| | amount of | | amount of | | amount of | |
| | cariprazine | Coefficient | cariprazine | Coefficient | cariprazine | Coefficient |
| Time | released | of variation | released | of variation | released | of variation |
| (min) | (%) | (%) | (%) | (%) | (%) | (%) |
| 3 | 104.1 | 4.8 | 61.2 | 3.1 | 37.6 | 3.1 |
| 6 | 106.0 | 1.6 | 66.2 | 1.4 | 41.2 | 3.2 |
| 10 | 104.1 | 5.3 | 69.0 | 1.3 | 44.1 | 2.7 |
| 15 | 101.2 | 1.8 | 70.7 | 1.0 | 47.0 | 2.7 |
| 30 | 102.6 | 4.7 | 77.1 | 1.7 | 53.4 | 2.0 |
| 60 | 100.5 | 2.4 | 86.3 | 3.1 | 63.5 | 2.0 |
| 120 | 102.8 | 0.8 | 96.5 | 4.3 | 78.8 | 1.5 |

TABLE 6

| | Experimental group D3 | | Experimental group D4 | | Experimental group D5 | |
|---|---|---|---|---|---|---|
| | (Dv50 = 10 μm) | | (Dv50 = 20 μm) | | (Dv50 = 40 μm) | |
| | Cumulative | | Cumulative | | Cumulative | |
| | amount of | | amount of | | amount of | |
| | cariprazine | Coefficient | cariprazine | Coefficient | cariprazine | Coefficient |
| Time | released | of variation | released | of variation | released | of variation |
| (min) | (%) | (%) | (%) | (%) | (%) | (%) |
| 3 | 26.4 | 0.5 | 20.7 | 0.7 | 16.5 | 1.9 |
| 6 | 30.7 | 0.3 | 23.7 | 0.5 | 19.5 | 2.0 |
| 10 | 33.4 | 0.2 | 26.2 | 1.8 | 22.2 | 1.6 |
| 15 | 36.0 | 0.5 | 28.5 | 0.9 | 24.8 | 2.4 |
| 30 | 41.3 | 0.5 | 33.5 | 1.7 | 30.0 | 3.3 |
| 60 | 48.2 | 0.9 | 39.6 | 1.4 | 37.0 | 4.4 |
| 120 | 58.5 | 3.0 | 49.4 | 3.0 | 47.4 | 5.4 |

FIG. 1 is a graph showing the cumulative amount of cariprazine released at different time points of the dissolution test for the injectable depot formulations of Experimental groups D1 to D5 and the Vraylar® oral capsule of Comparative group 1; whereas Tables 5 and 6 provides the numerical values and coefficient of variation thereof. As shown in FIG. 1 and Tables 5 and 6, in comparison with the Vraylar® oral capsule of Comparative Example 1 which comprises the cariprazine hydrochloride salt, the injectable depot formulations of Experimental groups D1 to D5 comprising cariprazine free base particles, which respectively have Dv50 of 2 μm, 8 μm, 10 μm, 20 μm and 40 μm, exhibited lower percentages of the cumulative amount of cariprazine released at the different time points of the dissolution test, suggesting that the cariprazine free base particles of the injectable depot formulation of Experimental groups D1 to D5 were released much more slowly compared with the cariprazine hydrochloride salt of the Vraylar® oral capsule of Comparative Example 1. To be specific, the cumulative amount of cariprazine free base particles released in each of Experimental groups D1 to D5 was less than 80% at the 15^{th} minute and was still less than 100% at the 120^{th} minute of the dissolution test (i.e., was not completely released after 120 minutes), whereas the cumulative amount of cariprazine hydrochloride salt released in the Comparative group 1 was greater than 100% within 10 minutes of the dissolution test. These results indicate that the injectable depot formulations of Experimental groups D1 to D5 each comprising the cariprazine free base particles exhibit improved sustained release behavior compared to the Vraylar® oral capsule of Comparative group 1 which comprises the cariprazine hydrochloride salt.

Referring again to FIG. 1 and Tables 5 and 6, in comparison with the injectable depot formulations of D1 to D4, the injectable depot formulation of D5, which comprises cariprazine free base particles having the largest Dv50 value, had the lowest percentages of the cumulative amount of cariprazine free base particles released over different time points of the dissolution test (i.e., the slowest dissolution rate), indicating that the injectable depot formulation comprising cariprazine free base particles with a relatively larger particle size had a relatively slower in vitro dissolution rate, and thus, demonstrates improved sustained release behaviour.

### Example 2. Evaluation of the Effect of Addition of Different Types of Suspending Agent on in Vitro Dissolution of Injectable Depot Formulation

In order to determine the effect of adding different types of suspending agents, i.e., sodium carboxymethyl cellulose, Povidone K30 and/or polyethylene glycol 4000, on the dissolution of the injectable depot formulation of the present disclosure, three different injectable depot formulations comprising cariprazine free base particles were prepared using the recipe shown in Table 7 below, so as to obtain injectable depot formulations of Experimental groups SA1, SA2, and SA3. Each of the injectable depot formulations of Experimental groups SA1, SA2 and SA3 was assayed, and then subjected to determination of particle size distribution, and the dissolution test so as to determine cumulative amount of cariprazine released. For comparison purpose, an injectable depot formulation comprising the same components as those of the injectable depot formulations of Example 1, was prepared and named as Experimental group D6 (concentration of cariprazine free base particles: 63 mg/mL). The injectable depot formulation of Experimental group D6 was also subjected to the aforesaid analysis and dissolution test.

The result of the particle size distribution analysis was shown in Table 8, and the result of the dissolution test was shown in FIG. 2 and Table 9.

TABLE 7

| | Experi- | Experi- | Experi- | Experi- |
|---|---|---|---|---|
| | mental | mental | mental | mental |
| Component | group | group | group | group |
| (amount) | D6 | SA1 | SA2 | SA3 |
| Cariprazine free base | 1.26 | 1.26 | 1.26 | 2.52 |
| particles (g) | | | | |
| Sodium carboxymethyl | 0.249 | 0.375 | 0.375 | — |
| cellulose (g) | | | | |
| Povidone K30 (g) | — | — | 0.180 | — |
| Polyethylene glycol 4000 | — | — | — | 1.80 |
| (g) | | | | |
| Mannitol (g) | 1.248 | 1.248 | 1.248 | 2.50 |
| Sodium dihydrogen | 0.0174 | 0.0174 | 0.0174 | 0.04 |
| phosphate monohydrate (g) | | | | |
| Disodium hydrogen | 0.0246 | 0.0246 | 0.0246 | 0.05 |
| phosphate anhydrous (g) | | | | |
| Water (for injection) | q.s. to | q.s. to | q.s. to | q.s. to |
| | 20 mL | 20 mL | 20 mL | 40 mL |

TABLE 8

| | Particle size distribution | | | |
|---|---|---|---|---|
| | Dv10 | Dv50 | Dv90 | Assay |
| Groups | (μm) | (μm) | (μm) | (%) |
| Experimental group D6 | 4.2 | 19.8 | 76.9 | 96.0 |
| Experimental group SA1 | 4.1 | 18.3 | 77.6 | 104.1 |
| Experimental group SA2 | 4.4 | 20.0 | 77.3 | 94.2 |
| Experimental group SA3 | 4.5 | 20.0 | 81.2 | 94.6 |

Referring to Table 8, the injectable depot formulations comprising cariprazine free base particles of Experimental groups SA1 to SA3 and Experimental group D6 showed similar particle size distribution profile. To be specific, in each group, the cariprazine free base particles had Dv50 value ranging from 18 μm to 20 μm; and the assay value thereof ranges from 90% to 105%.

TABLE 9

| | Cumulative amount of cariprazine released (%) | | | |
|---|---|---|---|---|
| Time | Experimental | Experimental | Experimental | Experimental |
| (min) | group D6 | group SA1 | group SA2 | group SA3 |
| 3 | 20.7 | 22.6 | 23.1 | 25.9 |
| 6 | 23.7 | 25.6 | 25.9 | 29.8 |
| 10 | 26.2 | 27.7 | 28.7 | 32.5 |
| 15 | 28.5 | 30.4 | 31.4 | 35.2 |
| 30 | 33.5 | 34.9 | 37.3 | 41.6 |
| 60 | 39.6 | 40.9 | 45.1 | 49.3 |
| 120 | 49.4 | 50.1 | 56.7 | 61.1 |

FIG. 2 is a graph showing the cumulative amount of cariprazine released at different time points of the dissolution test for the injectable depot formulations of Experimental groups SA1 to SA3 and Experimental group D6; whereas Table 9 provides the numerical values thereof. As shown in FIG. 2 and Table 9, the percentages of cumulative amount of cariprazine released at different time points of the dissolution test for the injectable depot formulation of Experimental group SA1 which contained sodium carboxymethyl cellulose as the suspending agent, the injectable depot formulation of Experimental group SA2 which contained sodium carboxymethyl cellulose as the first suspending agent and Povidone K30 as the second suspending agent, and injectable depot formulation of Experimental group SA3 which contained polyethylene glycol 4000 as the suspending agent, were not significantly different from those of the Experimental group D6, indicating that each of the injectable depot formulations of Experimental groups SA1 to SA3 and Experimental group D6 has similar sustained release profile. Therefore, addition of Povidone K30 or polyethylene glycol 4000 does not adversely affect the sustained released behavior of the injectable depot formulation comprising the cariprazine free base particles of the present disclosure.

### Example 3. Evaluation of the Effect of Addition of Different Types of Wetting Agent on in Vitro Dissolution of Injectable Depot Formulation

In order to determine the effect of adding different types of wetting agents, i.e., Polysorbate 80, Polysorbate 20 or Poloxamer 188, on the dissolution of the injectable depot formulation of the present disclosure, three different injectable depot formulations comprising cariprazine free base particles were prepared using the recipe shown in Table 10 below, so as to obtain injectable depot formulations of Experimental groups SA1+WA1, SA1+WA2, and SA1+WA3. In this example, the injectable depot formulation of Experimental group SA1 of Example 2 served as a control. Each of the injectable depot formulations of Experimental groups SA1+WA1 to SA1+WA3 and the Experimental group SA1 was assayed, and then subjected to determination of particle size distribution, and the dissolution test so as to determine cumulative amount of cariprazine released thereof.

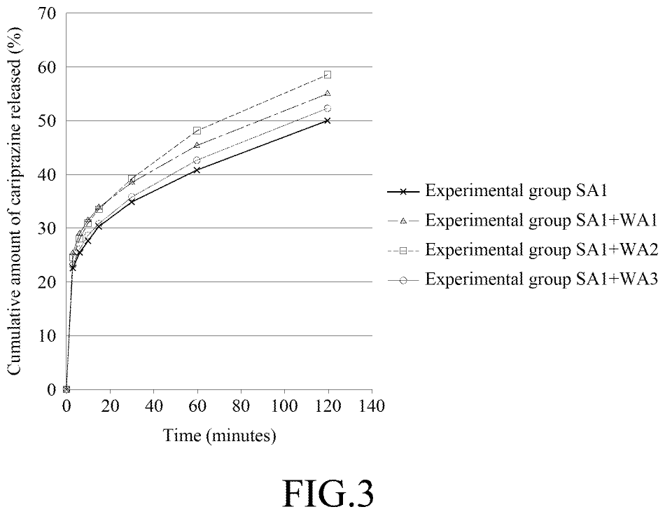

The result of the particle size distribution analysis was shown in Table 11, and the result of the dissolution test was shown in FIG. 3 and Table 12.

TABLE 10

| | | Experi- | Experi- | Experi- |
|---|---|---|---|---|
| | Experi- | mental | mental | mental |
| | mental | group | group | group |
| Component | group | SA1 + | SA1 + | SA1 + |
| (amount) | SA1 | WA1 | WA2 | WA3 |
| Cariprazine free base (g) | 1.26 | 1.26 | 1.26 | 1.26 |
| Sodium carboxymethyl | 0.375 | 0.375 | 0.375 | 0.375 |
| cellulose (g) | | | | |
| Polysorbate 80 (g) | — | 0.072 | — | — |
| Polysorbate 20 (g) | — | — | 0.6 | — |
| Poloxamer 188 (g) | — | — | — | 0.06 |
| Mannitol (g) | 1.248 | 1.248 | 1.248 | 1.248 |
| Sodium dihydrogen | 0.0174 | 0.0174 | 0.0174 | 0.0174 |
| phosphate monohydrate (g) | | | | |
| Disodium hydrogen | 0.0246 | 0.0246 | 0.0246 | 0.0246 |
| phosphate anhydrous (g) | | | | |
| Water (for injection) | q.s. to | q.s. to | q.s. to | q.s. to |
| | 20 mL | 20 mL | 20 mL | 20 mL |

TABLE 11

| | Particle size distribution | | | |
|---|---|---|---|---|
| | Dv10 | Dv50 | Dv90 | Assay |
| Groups | (μm) | (μm) | (μm) | (%) |
| Experimental group SA1 | 4.1 | 18.3 | 77.6 | 104.1 |
| Experimental group SA1 + WA1 | 3.1 | 11.8 | 53.6 | 104.2 |
| Experimental group SA1 + WA2 | 3.7 | 15.1 | 62.5 | 100.9 |
| Experimental group SA1 + WA3 | 3.8 | 16.5 | 68.5 | 96.0 |

Referring to Table 11, each of the injectable depot formulations of Experimental groups SA1+WA1, SA1+WA2, SA1+WA3 and Experimental group SA1 showed dispersion of cariprazine free base particles with Dv50 value ranging from 10 μm to 20 μm; and the assay value ranges from 95% to 105%. However, based on the Dv10, Dv50 and Dv90 values shown in Table 11, the injectable depot formulations of the Experimental groups SA1+WA1, SA1+WA2, SA1+WA3, which contained Polysorbate 80, Polysorbate 20 and Poloxamer 188, respectively serving as the wetting agent, comprise cariprazine free base particles with particle sizes smaller than those of the injectable depot formulation of the Experimental group SA1 which was prepared without the wetting agent.

TABLE 12

| | Cumulative amount of cariprazine released (%) | | | |
|---|---|---|---|---|
| | Experimental | Experimental | Experimental | Experimental |
| Time | group | group | group | group |
| (min) | SA1 | SA1 + WA1 | SA1 + WA2 | SA1 + WA3 |
| 3 | 22.6 | 25.3 | 24.6 | 23.2 |
| 6 | 25.6 | 29.0 | 28.1 | 26.2 |
| 10 | 27.7 | 31.8 | 31.0 | 28.8 |
| 15 | 30.4 | 34.1 | 33.6 | 30.9 |
| 30 | 34.9 | 38.9 | 39.3 | 35.9 |
| 60 | 40.9 | 45.7 | 48.2 | 42.7 |
| 120 | 50.1 | 55.3 | 58.6 | 52.4 |

FIG. 3 is a graph showing the cumulative amount of cariprazine released at different time points of the dissolution test for Experimental groups SA1+WA1 to SA1+WA3 and Experimental group SA1; whereas Table 12 provides the numerical values thereof. As shown in FIG. 3 and Table 12, the percentages of the cumulative amount of cariprazine released at different time points of the dissolution test for the injectable depot formulations of Experimental groups SA1+WA1, SA1+WA2 and SA1+WA3 were not significantly different from those of the injectable depot formulation of Experimental group SA1 which was prepared without the wetting agent. These results indicate that, each of the injectable depot formulations of Experimental groups SA1, SA1+WA1, SA1+WA2 and SA1+WA3 has similar sustained release profile. Therefore, addition of Polysorbate 80, Polysorbate 20, or Poloxamer 188 as the wetting agent does not adversely affect the sustained released behavior of the injectable depot formulation comprising the cariprazine free base particles of the present disclosure.

### Example 4. Evaluation of the Effect of Addition of Two Different Suspending Agents and One Single Wetting Agent on in Vitro Dissolution of Injectable Depot Formulation

In order to determine the effect of adding two different types of suspending agents (i.e., sodium carboxymethylcellulose and Povidone K30) and a single wetting agent, i.e., Polysorbate 80, Polysorbate 20 or lecithin, on the dissolution of the injectable depot formulations of the present disclosure, three different injectable depot formulations comprising cariprazine free base particles were prepared using the recipe shown in Table 13 below, so as to obtain injectable depot formulations comprising cariprazine free base particles of Experimental groups SA2+WA1, SA2+WA2, and SA2+WA4. In this example, the injectable depot formulation of Experimental group SA2 of Example 2 served as a control. Each of the injectable depot formulations of Experimental groups SA2+WA1 to SA2+WA4 and the Experimental group SA2 was assayed, and then subjected to determination of particle size distribution, and the dissolution test so as to determine cumulative amount of cariprazine released thereof.

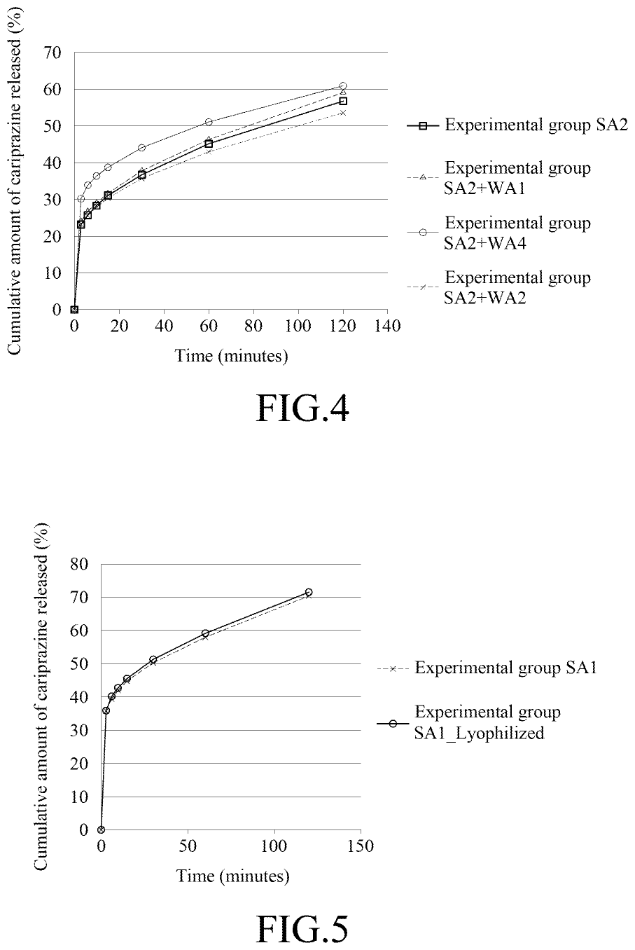

The result of the particle size distribution analysis was shown in Table 14, and the result of the dissolution test was shown in FIG. 4 and Table 15.

TABLE 13

| | | Experi- | Experi- | Experi- |
|---|---|---|---|---|
| | Experi- | mental | mental | mental |
| | mental | group | group | group |
| Component | group | SA2 + | SA2 + | SA2 + |
| (amount) | SA2 | WA1 | WA2 | WA4 |
| Cariprazine free base (g) | 1.26 | 1.26 | 1.26 | 1.26 |
| Sodium carboxymethyl | 0.375 | 0.375 | 0.375 | 0.375 |
| cellulose (g) | | | | |
| Povidone K30 (g) | 0.18 | 0.18 | 0.18 | 0.18 |
| Polysorbate 80 (g) | — | 0.072 | — | — |
| Polysorbate 20 (g) | — | — | 0.6 | — |
| Lecithin (g) | — | — | — | 0.18 |
| Mannitol (g) | 1.248 | 1.248 | 1.248 | 1.248 |
| Sodium dihydrogen | 0.0174 | 0.0174 | 0.0174 | 0.0174 |
| phosphate monohydrate | | | | |
| (g) | | | | |
| Disodium hydrogen | 0.0246 | 0.0246 | 0.0246 | 0.0246 |
| phosphate anhydrous (g) | | | | |
| Water (for injection) | q.s. to | q.s. to | q.s. to | q.s. to |
| | 20 mL | 20 mL | 20 mL | 20 mL |

TABLE 14

| | Particle size distribution | | | |
|---|---|---|---|---|
| | Dv10 | Dv50 | Dv90 | Assay |
| Groups | (μm) | (μm) | (μm) | (%) |
| Experimental group SA2 | 4.4 | 20.0 | 77.3 | 94.2 |
| Experimental group SA2 + WA1 | 3.9 | 16.3 | 66.2 | 92.9 |
| Experimental group SA2 + WA2 | 4.4 | 19.9 | 80.9 | 94.5 |
| Experimental group SA2 + WA4 | 3.3 | 13.0 | 57.1 | 109.5 |

Referring to Table 14, each of the injectable depot formulations of Experimental groups SA2+W1, SA2+W2, SA2+W4 and Experimental group SA2 showed dispersion of cariprazine free base particles with Dv50 value ranging from 10 μm to 20 μm; and the assay value ranging from 90% to 110%. However, based on the Dv10, Dv50 and Dv90 values shown in Table 14, the injectable depot formulation of the Experimental group SA2+WA4, which contained lecithin as the wetting agent, comprises cariprazine free base particles with particle size smaller than those of the injectable depot formulations of the Experimental group SA2 (without the wetting agent), and also those of the Experimental groups SA2+WA1 and SA2+WA2 which contained Polysorbate 80 and Polysorbate 20, respectively serving as the wetting agent.

TABLE 15

| | Cumulative amount of cariprazine released (%) | | | |
|---|---|---|---|---|
| | Experimental | Experimental | Experimental | Experimental |
| Time | group | group | group | group |
| (min) | SA2 | SA2 + WA1 | SA2 + WA2 | SA2 + WA4 |
| 3 | 23.1 | 24.1 | 23.0 | 30.0 |
| 6 | 25.9 | 26.9 | 25.3 | 33.7 |
| 10 | 28.7 | 29.2 | 27.9 | 36.4 |
| 15 | 31.4 | 31.9 | 30.3 | 38.6 |
| 30 | 37.3 | 38.0 | 35.7 | 43.8 |
| 60 | 45.1 | 46.4 | 42.9 | 50.9 |
| 120 | 56.7 | 59.3 | 53.6 | 60.9 |

FIG. 4 is a graph showing the cumulative amount of cariprazine released at different time points of the dissolution test for Experimental groups SA2+WA1 to SA2+WA4 and Experimental group SA2; whereas Table 15 provides the numerical values thereof. As shown in FIG. 4 and Table 15, the percentages of the cumulative amount of cariprazine released at different time points of the dissolution test for the injectable depot formulation of Experimental group SA2+WA1 which contained Polysorbate 80 as the wetting agent, the injectable depot formulation of Experimental group SA2+WA2 which contained Polysorbate 20 as the wetting agent, and the injectable depot formulation of Experimental group SA2+WA4 which contained lecithin as the wetting agent, were not significantly different from those of the injectable depot formulations of Experimental group SA2 which contained the same suspending agents but without the wetting agent. These results indicate that, each of the injectable depot formulations of Experimental groups SA2, SA2+WA1, SA2+WA2, and SA2+WA4 has similar sustained release profile. Therefore, addition of polysorbate or lecithin does not adversely affect the sustained released behavior of the injectable depot formulation comprising the cariprazine free base particles of the present disclosure.

### Example 5. Evaluation of the Effect of Lyophilization Process on in Vitro Dissolution of Injectable Depot Formulation

In order to determine whether lyophilized form of the injectable depot formulation affected the stability thereof, in this example, the injectable depot formulation of Experimental group SA1 of Example 2 (concentration of cariprazine free base particles: 63 mg/mL) was subjected to a lyophilization process performed according to the steps and conditions listed in Table 16 below, so as to obtain injectable depot formulation of Experimental group SA1_Lyophilized. The injectable depot formulation of Experimental group SA1 of Example 2, which was not subjected to the lyophilization process, served as a control. Each of the injectable depot formulations of Experimental groups SA1_Lyophilized and the Experimental group SA1 was assayed, and then subjected to determination of particle size distribution, and the dissolution test as described in Example 1, so as to determine cumulative amount of cariprazine released thereof.

The result of the particle size distribution analysis was shown in Table 17, and the result of the dissolution test was shown in FIG. 5 and Table 18.

TABLE 16

| | | Temperature | | Time period |
|---|---|---|---|---|
| Step | | (° C.) | Pressure | (hour) |
| 1 | Freezing | −40 | Ambient | 1 |
| 2 | Extra freezing | −40 | Ambient to | As required |
| | | | 100 mTorr | |
| 3 | Primary drying | 5 | 100 mTorr | 2880 |
| 4 | Secondary | 30 | 400 mTorr | 0.25 |
| | drying | | | |

TABLE 17

| | Particle size distribution | | | |
|---|---|---|---|---|
| | Dv10 | Dv50 | Dv90 | Assay |
| Group | (μm) | (μm) | (μm) | (%) |
| Experimental group SA1 | 2.8 | 11.0 | 46.5 | 98.6 |
| Experimental group SA1_Lyophi- | 2.7 | 10.0 | 41.6 | 101.0 |
| lized | | | | |

Referring to Table 17, the injectable depot formulations comprising cariprazine free base particles of Experimental groups SA1 and Experimental group SA1_Lyophilized showed almost similar particle size distribution profile. To be specific, the cariprazine free base particles dispersed had Dv50 value ranging from 10 μm to 11 μm; and with the assay value ranging from 95% to 101%.

TABLE 18

Cumulative amount of cariprazine released (%)

| Time | Experimental | Experimental group |
|---|---|---|
| (min) | group SA1 | SA1_lyophilized |
| 3 | 35.7 | 36.1 |
| 6 | 39.5 | 40.2 |
| 10 | 42.0 | 42.6 |
| 15 | 44.7 | 45.4 |
| 30 | 50.3 | 51.2 |
| 60 | 58.0 | 58.9 |
| 120 | 70.4 | 71.4 |

FIG. 5 is a graph showing the cumulative amount of cariprazine released at different time points of the dissolution test for Experimental group SA1_Lyophilized and Experimental group SA1; whereas Table 18 provides the numerical values thereof. As shown in FIG. 5 and Table 18, the percentages of the cumulative amount of cariprazine released from the injectable depot formulations of Experimental group SA1_Lyophilized and the Experimental group SA1 at different time points of the dissolution test were not significantly different from one another, indicating that the lyophilization process did not adversely affect the release of cariprazine free base particles from the injectable depot formulation, and the lyophilized injectable depot formulation remains stable.

### Example 6. Evaluation of the Effect of Different Concentrations of Cariprazine Free Base Particles on in Vitro Dissolution of Injectable Depot Formulation

In order to determine the effect of increased amount and/or concentration of cariprazine free base particles on the dissolution of the injectable depot formulation of the present disclosure, four different injectable depot formulations comprising cariprazine free base particles were prepared using the recipe shown in Table 19 below and in accordance with the procedures described in Example 1, so as to obtain injectable depot formulations of Experimental groups C1, C2, C3 and C4 having concentrations of 84 mg/mL, 187 mg/mL, 336 mg/mL and 481 mg/mL, respectively. In addition, the injectable depot formulation of the Experimental group C4 was further subjected to the lyophilization process described in Example 5, followed by reconstituting the lyophilized injectable depot formulation with appropriate amounts of water, thereby obtaining the injectable depot formulations of Experimental group C5 and Experimental group C6 having concentrations of 548 mg/mL and 618 mg/mL, respectively. Thereafter, each of the injectable depot formulations of Experimental groups C1 to C6 was subjected to the dissolution test as described in Example 1 so as to determine cumulative amount of cariprazine released thereof.

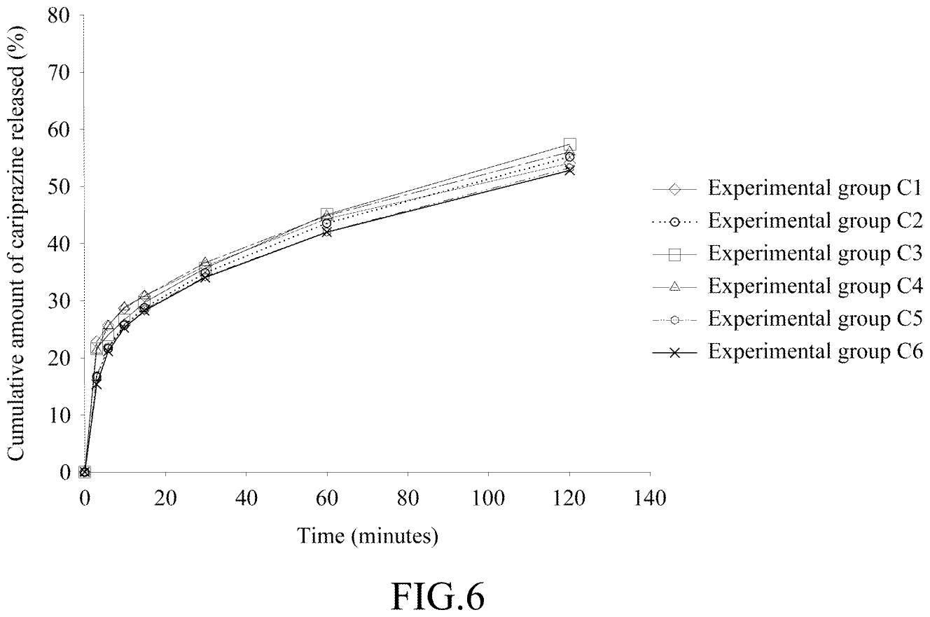

The result of the dissolution test was shown in FIG. 6 and Table 20.

TABLE 19

| | Experi- | Experi- | Experi- | Experi- |
|---|---|---|---|---|
| | mental | mental | mental | mental |
| | group C1 | group C2 | group C3 | group C4 |
| Component | (84 | (187 | (336 | (481 |
| (amount) | mg/mL) | mg/mL) | mg/mL) | mg/mL) |
| Cariprazine free base (g) | 1.68 | 4.2 | 8.4 | 9.0 |
| Sodium | 0.1664 | 0.1664 | 0.1664 | 0.1664 |
| carboxymethylcellulose | | | | |
| (g) | | | | |
| Mannitol (g) | 0.832 | 0.832 | 0.832 | 0.832 |
| Sodium dihydrogen | 0.0116 | 0.0116 | 0.0116 | 0.0116 |
| phosphate monohydrate | | | | |
| (g) | | | | |
| Disodium hydrogen | 0.0165 | 0.0165 | 0.0165 | 0.0165 |
| phosphate anhydrous (g) | | | | |
| Water (for injection) | q.s. to | q.s. to | q.s. to | q.s. to |
| | 20 mL | 22.5 mL | 25 mL | 20 mL |

TABLE 20

| | Cumulative amount of cariprazine released (%) | | | | | |
|---|---|---|---|---|---|---|
| | Experimental | Experimental | Experimental | Experimental | Experimental | Experimental |
| Time | group C1 | group C2 | group C3 | group C4 | group C5 | group C6 |
| (min) | (84 mg/mL) | (187 mg/mL) | (336 mg/mL) | (481 mg/mL) | (548 mg/mL) | (618 mg/mL) |
| 3 | 22.5 | 21.5 | 21.1 | 21.9 | 16.6 | 15.4 |
| 6 | 25.6 | 23.8 | 23.7 | 26.2 | 22.0 | 21.2 |
| 10 | 28.6 | 26.5 | 26.9 | 29.0 | 25.5 | 25.3 |
| 15 | 30.9 | 28.8 | 29.4 | 31.1 | 28.4 | 28.2 |
| 30 | 36.2 | 35.0 | 35.3 | 37.1 | 34.1 | 34.0 |
| 60 | 44.2 | 43.5 | 44.9 | 45.0 | 41.8 | 41.9 |
| 120 | 53.8 | 55.1 | 57.3 | 56.0 | 53.1 | 52.8 |

FIG. 6 is a graph showing the cumulative amount of cariprazine released at different time points of the dissolution test for Experimental groups C1 to C6; whereas Table 20 provides the numerical values thereof. As shown in FIG. 6 and Table 20, the percentages of the cumulative amount of cariprazine released from the injectable depot formulations of Experimental groups C1 to C6 at different time points of the dissolution test were not significantly different from one another, indicating that the increased concentration of the cariprazine free base particles did not adversely affect the release thereof from the injectable depot formulations, and that the injectable depot formulation of the present disclosure has a high drug load.

### Example 7. Evaluation of the Effect Of Different Dv50 of Cariprazine Free Base Particles on in Vivo Detection of Cariprazine in Blood Plasma

Since the results in Example 1 demonstrate that the injectable depot formulation comprising the cariprazine free base particles of the present disclosure had a dissolution rate which is lower than that of the Vraylar® oral capsule comprising cariprazine hydrochloride salt, the applicants performed pharmacokinetic studies using animal model to determine the effect of different Dv50 of the cariprazine free base particles on in vivo dissolution thereof in blood plasma of rats, and also to determine the difference in the pharmacokinetic profiles between the rats administered intramuscularly with the injectable depot formulation comprising the cariprazine free base particles of the present disclosure and the rats administered orally with the Vraylar® capsule.

### A. First Pharmacokinetic Study Using Injectable Depot Formulation of the Present Disclosure

The four injectable depot formulations of this example, as shown in Table 21 below, were similar to the injectable depot formulations of Experimental groups D1, D3, D4 and D5 as described in Example 1, and were prepared using the procedures described in Example 1.

TABLE 21

| Injectable depot formulation | Dv50 (μm) |
|---|---|
| Experimental group D1 | 2 |
| Experimental group D3 | 10 |
| Experimental group D4 | 20 |
| Experimental group D5 | 40 |

The Wistar rats (6 to 8 weeks old) were housed in an animal room with an independent air conditioning system under the following laboratory conditions: an alternating 12-hour light and 12-hour dark cycle, a temperature maintained at 23° C.±2° C., and a relative humidity maintained at 40% to 70%. The rats were provided with water and fed ad libitum. All experimental procedures involving the experimental rats were in compliance with the legal provision of the Animal Protection Act of Taiwan, and were carried out according to the guidelines of the Animal Care Committee of the Council of Agriculture, Taiwan.

The rats were randomly divided into four groups, namely, Experimental group D1, Experimental group D3, Experimental group D4, and Experimental group D5, in which the number of rats in each of the Experimental groups D1 and D3 was 6, and the number of rats in each of the Experimental groups D4 and D5 was 4.

The rats in the Experimental group D1 to D5 were intramuscularly administered with the injectable depot formulations of Experimental group D1 to D5, respectively. The intramuscular administration was performed by injecting a single dose of an aqueous suspension comprising cariprazine free base particles into the thigh muscle of the rats at a concentration of 28 mg/kg per rat. For the rats in each of the Experimental groups D1, D3, D4 and D5, blood samples were collected from the tail vein of each rat on the 1^{st} day, (i.e., 10^{th} and 30^{th} minute, and 1^{st}, 2^{nd}, 3^{rd}, 5^{th}, 7^{th}, 10^{th} and 24^{th} hour post-administration), and on the 3^{rd}, 5^{th}, 7^{th}, 10^{th}, 14^{th}, 21^{th}, and 28^{th} day, and then were subjected to measurement of mean plasma concentration using QTRAP® 5500 LC-MS/MS system (AB Sciex). For the rats in the Experimental group D3, blood samples were also collected on the 35^{th} and 42^{nd} day post-administration, whereas for the rats in the Experimental groups D4 and D5, blood samples were also collected on the 35^{th}, 42^{nd}, 49^{th} and 56^{th} day post-administration.

TABLE 22

| | Experimental | Experimental | Experimental | Experimental |
|---|---|---|---|---|
| Pharmacokinetic | group D1 | group D3 | group D4 | group D5 |
| parameters | (Day₀₋₂₈) | (Day₀₋₄₂) | (Day₀₋₅₆) | (Day₀₋₅₆) |
| T_{1/2} (hour) | 77.8 ± 14.8 | 113.7 ± 25.2 | 141.6 ± 28.8 | 196.8 ± 48.0 |
| Cₘₐₓ (ng/mL) | 83.2 ± 8.5 | 40.0 ± 11.4 | 39.9 ± 6.6 | 28.8 ± 9.8 |
| Tₘₐₓ (hour) | 112.0 ± 19.6 | 104.0 ± 39.2 | 120 ± 0.0 | 72 ± 55.2 |
| AUCₐₗₗ (h*ng/mL) | 13256.8 ± 950.8 | 12369.1 ± 2077.3 | 15482.4 ± 2520 | 11793.6 ± 2808 |

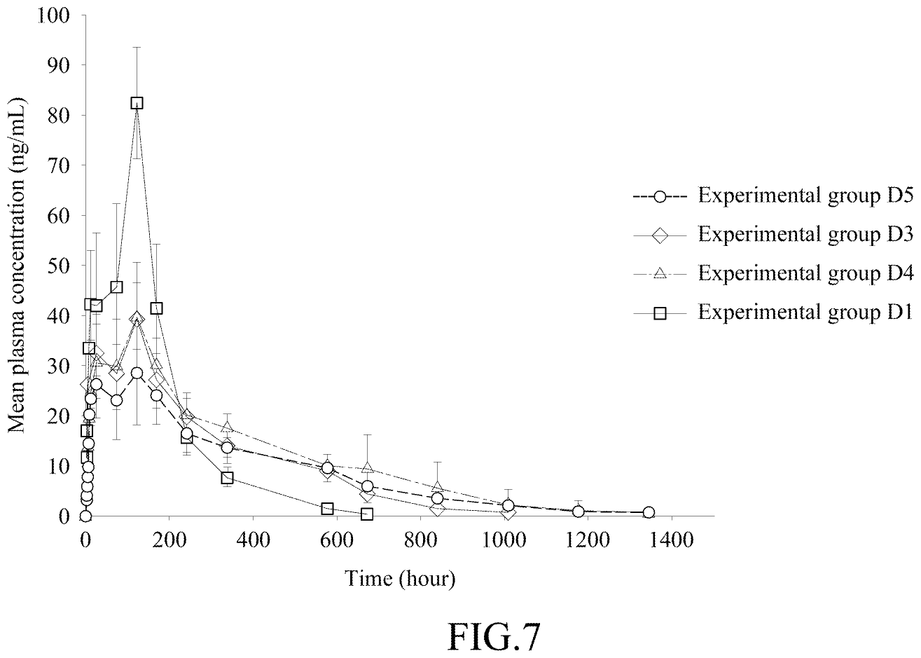

FIG. 7 is a graph showing the mean plasma concentration of cariprazine in the rats of the Experimental groups D1, D3, D4 and D5 following intramuscular administration of the injectable depot formulations comprising cariprazine free base particles of the present disclosure over an entire time period in a first pharmacokinetic study; whereas Table 22 provides the parameters determined from the first pharmacokinetic study. As shown in FIG. 7, in each of the Experimental groups D1, D3, D4 and D5, after intramuscular administration of the injectable depot formulations comprising cariprazine free base particles, the mean plasma concentration of cariprazine gradually reached a peak value only after 100 hours and then decreased slowly, and cariprazine was continuously detected in the plasma of the rats throughout the 28-day time period of the first pharmacokinetic study. In particular, cariprazine was still detected in the plasma of the rats of the Experimental group D3 until the 42^{nd} day post-administration, and was still detected in the plasma of the rats of the Experimental groups D4 and D5 until the 56^{th} day post-administration. In addition, as shown in Table 22, the T_{1/2} value increased from Experimental groups D1, D3, D4 and D5, corresponding to an increase in the size of the cariprazine free base particles in these groups; while the high AUCₐₗₗ value determined in each of the Experimental groups D1, D3, D4 and D5 suggests that the cariprazine free base particles released from the injectable depot formulations have good bioavailability. These results indicate that the injectable depot formulations comprising cariprazine free base particles with Dv50 ranging from 2 μm to 40 μm demonstrate in vivo sustained release behavior for at least 28 days.

### B. Second Pharmacokinetic Study Using Vraylar® Capsule

Six Wistar rats (6 to 8 weeks old) were grouped to form Comparative group 1. Vraylar® capsule were dissolved in 0.1% acetic acid to prepare an aqueous suspension containing cariprazine hydrochloride salt, which was then orally administered to the rats of the Comparative group 1, via oral gavage, at a concentration of 1 mg/kg per rat. Blood samples were collected from the tail vein of each rat on the 10^{th} and 30^{th} minute, and on the 1^{st}, 2^{nd}, 3^{rd}, 5^{th}, 7^{th}, 10^{th} and 24^{th} hour post-administration), and then were subjected to measurement of mean plasma concentration using the procedures as described in section A above.

TABLE 23

| Pharmacokinetic | Comparative group 1 |
|---|---|
| parameters | (Hour₀₋₂₄) |
| T_{1/2} (hour) | 3.28 ± 0.33 |
| Cₘₐₓ (ng/mL) | 19.6 ± 3.69 |
| Tₘₐₓ (hour) | 2.67 ± 0.52 |
| AUCₐₗₗ (h*ng/mL) | 138.29 ± 33.45 |

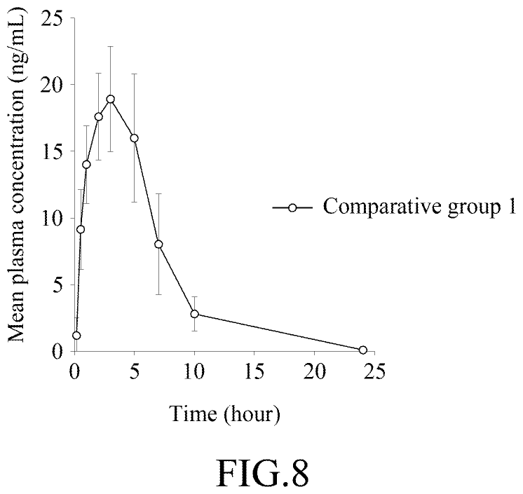

FIG. 8 is a graph showing the mean plasma concentration of cariprazine in the rats of the Comparative group 1 following oral administration of Vraylar® capsule over a 24-hour time period in a second pharmacokinetic study; whereas Table 23 provides the parameters determined from the second pharmacokinetic study. As shown in FIG. 8, after administration of the Vraylar® capsule comprising cariprazine hydrochloride salt, the mean plasma concentration of cariprazine reached a peak value within 2 hours and then decreased rapidly within 10 hours; and cariprazine was no longer detected in the plasma of the rats of the Comparative group 1 after 24 hours post-administration. In addition, sustained released behavior was not readily observed following oral administration of the Vraylar® capsule because the T_{1/2} value of the Comparative group 1 (see Table 23) was relatively smaller than those of the Experimental groups D1, D3, D4 and D5 (see Table 22). This results indicate that, compared with the injectable depot formulation comprising cariprazine free base particles, which was administered intramuscularly to the rats as described in section A of this example, the orally administered Vraylar® capsule comprising cariprazine hydrochloride salt, fails to demonstrate sustained release behavior.

Taken together, these results demonstrate that the size of cariprazine free base particles is a critical factor which modulates the sustained release behavior of the injectable depot formulations comprising the cariprazine free base particles of the present disclosure. The increase in the size of the cariprazine free base particles results in slower release thereof from the injectable depot formulation, leading to a slower in vitro dissolution rate. Since the injectable depot formulation comprising cariprazine free base particles of the present disclosure also demonstrates a controlled, sustained release effect in vivo, it is expected to be useful as an anti-psychotic drug with reduced dosing frequency.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An injectable depot formulation comprising, cariprazine free base particles and a pharmaceutically acceptable carrier, wherein the cariprazine free base particles have a median particle size by volume (Dv50) ranging from 0.5 μm to 100 μm.

2. The injectable depot formulation as claimed in claim 1, wherein the cariprazine free base particles have a median particle size by volume (Dv50) ranging from 2 μm to 40 μm.

3. The injectable depot formulation as claimed in claim 1, wherein the cariprazine free base particles are present in an amount ranging from 4 wt % to 65 wt %, based on a total weight of the injectable depot formulation.

4. The injectable depot formulation as claimed in claim 1, wherein the pharmaceutically acceptable carrier comprises at least one of a suspending agent, a buffering agent, and a tonicity agent.

5. The injectable depot formulation as claimed in claim 4, wherein the suspending agent is present in an amount ranging from 0.2 wt % to 10 wt %, based on the total weight of the injectable depot formulation.

6. The injectable depot formulation as claimed in claim 4, wherein the suspending agent comprises a cellulose derivative, povidone, polyethylene glycol, or combinations thereof.

7. The injectable depot formulation as claimed in claim 6, wherein the cellulose derivative comprises carboxymethyl cellulose, sodium carboxymethyl cellulose, and methyl cellulose.

8. The injectable depot formulation as claimed in claim 4, wherein the buffering agent is present in an amount ranging from 0.01 wt % to 2 wt %, based on the total weight of the injectable depot formulation.

9. The injectable depot formulation as claimed in claim 4, wherein the buffering agent comprises sodium phosphate monobasic, disodium hydrogen phosphate, citric acid, sodium citrate, or combinations thereof.

10. The injectable depot formulation as claimed in claim 4, wherein the tonicity agent is present in an amount ranging from 0.01 wt % to 6.5 wt %, based on the total weight of the injectable depot formulation.

11. The injectable depot formulation as claimed in claim 4, wherein the tonicity agent comprises mannitol, glucose, sucrose, trehalose, and sodium chloride.

12. The injectable depot formulation as claimed in claim 4, wherein the pharmaceutically acceptable carrier further comprises a wetting agent.

13. The injectable depot formulation as claimed in claim 12, wherein the wetting agent is present in an amount ranging from 0.01 wt % to 3 wt %, based on the total weight of the injectable depot formulation.

14. The injectable depot formulation as claimed in claim 12, wherein the wetting agent comprises polysorbate, poloxamer, lecithin, sorbitan monolaurate, and polyoxyethylene fatty acid esters.

15. The injectable depot formulation as claimed in claim 4, wherein the pharmaceutically acceptable carrier further comprises a pH-adjusting agent.

16. The injectable depot formulation as claimed in claim 15, wherein the pH-adjusting agent is present in an amount ranging from 0.01 wt % to 1 wt %, based on the total weight of the injectable depot formulation.

17. The injectable depot formulation as claimed in claim 15, wherein the pH-adjusting agent comprises sodium hydroxide and hydrogen chloride.

18. A method for treating psychosis, cognitive impairment accompanying schizophrenia, bipolar disorder, acute mania, mild-to-moderate cognitive deficits, dementia, psychotic states associated with dementia, psychotic depression, mania, paranoid and delusional disorders, dyskinetic disorders, neuroleptics-induced parkinsonism, tardive dyskinesia, eating disorders, attention deficit disorder, hyperactivity disorders in children, depression, anxiety, sexual dysfunction, sleep disorders, emesis, aggression, autism, major depressive disorder or drug abuse, comprising the step of administering an injectable depot formulation of claim 1 to a subject in need thereof.

19. An injectable depot formulation comprising cariprazine free base particles and a pharmaceutically acceptable carrier, wherein the cariprazine free base particles have a median particle size by volume (Dv50) ranging from 0.5 μm to 100 μm, wherein the pharmaceutically acceptable carrier comprises at least one of a suspending agent, a buffering agent, a tonicity agent, and combinations thereof, wherein the suspending agent comprises cellulose derivative, povidone, and polyethylene glycol, the buffering agent comprises sodium phosphate monobasic, disodium hydrogen phosphate, citric acid, and sodium citrate, and the tonicity agent comprises mannitol, glucose, sucrose, trehalose, and sodium chloride.

20. The injectable depot formulation as claimed in claim 19, wherein the cariprazine free base particles have a median particle size by volume (Dv50) ranging from 8 μm to 40 μm.
